(19) United States
(12) Patent Application Publication
(10) Pub. No.: US 2023/0386655 A1
(43) Pub. Date: Nov. 30, 2023

(54) CLOUD-BASED, SCALABLE, ADVANCED ANALYTICS PLATFORM FOR ANALYZING COMPLEX MEDICAL RISK DATA AND PROVIDING DEDICATED ELECTRONIC TRIGGER SIGNALS FOR TRIGGERING RISK-RELATED ACTIVITIES IN THE CONTEXT OF MEDICAL RISK-TRANSFER, AND METHOD THEREOF

(71) Applicant: Swiss Reinsurance Company Ltd., Zürich (CH)

(72) Inventors: Nikos KOUVARAS, Horgen (CH); Aman CHAWLA, Kolkata (IN); Charilaos TSAROUCHAS, Gockhausen (CH)

(73) Assignee: Swiss Reinsurance Company Ltd., Zürich (CH)

(21) Appl. No.: 18/446,949

(22) Filed: Aug. 9, 2023

Related U.S. Application Data
(63) Continuation of application No. PCT/EP2022/062326, filed on May 6, 2022.

(30) Foreign Application Priority Data
May 7, 2021 (CH) 00519/2021

(51) Int. Cl. G16H 40/20; G16H 50/20; G06Q 40/08

(52) U.S. Cl. CPC G16H 40/20; G16H 50/20; G06Q 40/08

(57) ABSTRACT

Proposed is a cloud-based, scalable, advanced analytics platform and/or anomaly detection system 1 for analyzing complex medical risk data and providing dedicated electronic trigger signals, inter alia, applicable for triggering risk-related activities or providing expert-system-based insights for medical risk-transfer. The digital-based system can, inter alia, be based on measurements based on evolving real-world measuring parameters associated with complex medical or clinical risk-related measuring parameter values and data. The present invention further relates to pattern anomaly detection and more specifically to a method and apparatus for performing a multi-domain anomaly pattern definition and detection, in particular in the filed bio-surveillance.

### CROSS-REFERENCE TO RELATED APPLICATIONS

This application is a continuation of and claims benefit under 35 U.S.C. § 120 to International Application No. PCT/EP2022/062326 filed on May 6, 2022, which is based upon and claims priority to Swiss Application No. 00519/2021, filed May 7, 2021, the entire contents of each of which are hereby incorporated herein by reference.

### FIELD OF THE INVENTION

The present invention relates to a cloud-based, scalable, advanced analytics platform for analyzing complex medical risk data and providing dedicated electronic trigger signals for triggering risk-related activities or providing expert-system-based insights for medical risk-transfer. The digital-based system can, inter alia, be based on measurements based on evolving real-world measuring parameters associated with complex medical or clinical measuring parameter values and data. The present invention further relates to pattern anomaly detection and more specifically to a method and apparatus for performing a multi-domain anomaly pattern definition and detection, in particular in the filed bio-surveillance. Thus, the present invention also relates to automated detection of the so-called Fraud, Waste and Abuse (WAF) cases, and automated and/or electronic systems solving trying to solve this problem notorious about its technical complexity.

### BACKGROUND OF THE INVENTION

A great amount of money is spent every year on health care across developed and developing countries. Such costs are expected to continue rising as populations age and medical treatments evolve demanding more sophisticated equipment and the implementation of new technologies. Hence, a key challenge facing the healthcare industry lies in delivering quality health care while keeping costs under control. In addition, Fraud, Waste and Abuse (FWA) cases represent a significant component of medical cost inflation. FWA is estimated to account for ca. 3-10% of healthcare costs yearly (see e.g. fraud prevention study from the National Health Care Anti-Fraud Association (NHCAA)). Across the medical insurance community, appetite to address FWA is increasing but so is the technical complexity to tackle them.

Further, in the nowadays increasingly digitized world, billions of digital records are diligently collected by medical insurers' data capturing systems every day. Policies' parameters, claims' data, and distribution networks generate increasingly larger sets of complex and diverse data. Leveraging these data sets is key to automated managing portfolio performance and informing business decisions based on expert system operations. However, most industries, in particular risk-transfer/insurance technologies, do not take advantage of these powerful technically accessible insights, as data is usually kept in data silos that do not intercommunicate. Putting this data to work requires combining data, technology, and expertise to slice and dice chunks of Big Data and draw actionable insights. Insights on emerging trends, changes in consumer behavior, pricing enhancement potential and new product designs can bring new business opportunities to light and safeguard against abusive and adverse behavior across the medical insurance ecosystem. Thus, there is a great technical need for an automated system, allowing to provide automated big data analysis techniques particularly directed to data recognition and processing in the medical portfolio data maze.

Further technical challenges are in providing technically automated devices able to analyze medical reimbursement claims behavior and performance early in the process, which is crucial to address real-time reacting systems' issues proactively. Claims processing can have significant impact on medical portfolio management and profitability and solvency while also playing an instrumental role in defining customer experience and overall competitiveness. However, making sense of these humongous and complex medical insurance datasets is a daunting, tedious, lengthy, and error-prone exercise. Amid this immense data maze, overlooking a valuable piece of information can put the appropriate claims decision at risk. By the same token, excessive scrutiny may slow down the process and lead to customer dissatisfaction. Furthermore, increasing competition is driving the need to create efficiencies and to strike the right balance between the cost of claims and an optimized customer experience. But issues with leakages, abuse and medical identity theft are growing areas of concern that call upon the identification of outlier patterns to effect risk mitigation strategies. Also, these growing risks demand more emphasis be put on pricing models based on risk categorization to secure sustainable growth. There is a need to have a system technically enabled to dramatically transform the medical reimbursement process to provide medical insurers and automated risk-transfer systems with machine-based intelligent and dynamic data analytics with actionable expert-system based insights in a matter of minutes.

In general, in many fields of technology, it is often a demand to make precise assessment and/or predictions regarding the evolving operation or status of living objects or other real world physical systems, such as time characteristics and temporal behavior of products, human beings or animals based on measured parameters and sensory data, for example for precise personalized and predictive medicine (e.g. telematics based), floating short or long scale risk assessment and measurements of the physical real-world (living) objects. Specifically, in the healthcare technology, rising healthcare costs and concerns about increasing the availability and quality of healthcare have led to an increased use of predictive model-based electronic expert-systems to identify those patients most likely to have a need for specific types of healthcare services. The ability to identify predictors of different health problems and diseases and apply them to patient populations can be important in determining where patients should be directed for additional care. Predictors are useful in identifying patients likely to benefit from various intervention and prevention programs so that future health-care problems are avoided or minimized and related costs are reduced. U.S. Pat. No. 7,725,329 describes one system and method for predicting a person's future health status based on various clinical measuring parameters. Using medical and pharmacy claim data from health benefits providers, the presence of clinical conditions is determined and based on the clinical conditions, a person's future health status is predicted. Although the presence or absence of various clinical conditions is important to predicting a person's health status, consideration of other factors may increase the accuracy of the predictive model.

There is a need for an improved automated predictive model-based systems, in particular simulation-based measuring systems, for measuring real-time performance of a portfolio of health-risk-related living objects based on real-world sensory links and measurement, adaptive claim triage, complex decision and trigger signaling, intelligent machine-learning based assessments and measuring, in particular health risk assessments, advanced machine-based analytics capabilities, work-flow allocation and adaption and reliable, robust automated expert insights and advices. Further, there is a need for an automated dynamic portfolio optimizer capturing health risks that delivers automated, faster, and better insights to users.

Further, health related measuring parameters and data are complex and heterogeny, thus though an analysis of applicable claim data is helpful in understanding, and, therefore, controlling health care risks, portfolios, and health care costs, performing an analysis is typically not a straightforward task. For example, administrative claim data normally does not contain information on an insured's height and weight, and yet obesity (as measured by the Body Mass Index) is a key contributor to health and wellness and, therefore, health care costs. Many health conditions are related to obesity and so it is useful to understand the levels or degrees of obesity present in a population. In this example, the levels or degrees of obesity in an insured population may influence the claims that are made under a health care plan and help a sponsor understand factors that may be contributing to the costs. However, without the height and weight data for the individuals covered by the plan, it is difficult to determine the level or degree of obesity among the individuals, and therefore, whether health care costs under the plan are potentially attributable to obesity-related health conditions. There is a need for a computerized system and method for estimating the presence and levels or degrees of medial risks driving parameters, as e.g. obesity in an insured population, inter alia, using claims data or other accessible measuring parameters, as e.g. clinical parameters.

The prior art document US 2018/239870 A1 discloses a system for automatically identifying and addressing potential healthcare-based fraud. The system identifies potential healthcare-based fraud associated with potentially suspicious healthcare providers, patients, and/or claim submissions by acquiring data associated with a healthcare provider, patient, and/or claim submission, by applying the data to one or more predictive model structures to generate one or more risk score measures identifying potential health-care-based fraud, and perform risk reduction actions based on the risk scores. Further, US 2020/005080 A1 discloses a systems based on machine-learning structures to predict recovery rate measures/score measures for occurring claims, predict priority scores for the claims, and automatically prioritizing initiation of claim settlement procedures based on the predicted measures, and/or signaling a user interface based on the prioritization. US 2021/103991 A1 discloses a system for automated medical malpractice risk-transfer underwriting based on processed value-based care data. A machine-learning-based predictive structure is trained to predict a future probability measures of an occurrence of a medical malpractice claim from a retrieved data set comprising value-based care data and social factor data. The data set is inputted into the trained machine-learning based predictive structure. A risk score measure is predicted measuring a probability value for a future occurrence of a medical malpractice claim based on the input data set using the trained machine-learning based predictive structure. A premium amount value for medical malpractice risk-transfer is determined based on the predicted risk score measure. The predictive modeling structure can also be used to predict stop loss risk and determine a combined premium amount value for medical malpractice and stop loss risk-transfer. Finally, US 2016/055589 A1 discloses a forecast system predicting and automatically identifying claims that have a high likelihood of exceeding a predetermined limitation in a given threshold value for excess of workers' compensation risk-transfer. The system automatically signals and generates possible intervention strategies to mitigate potentially occurring excess claims costs. The system processes associated claims, payment, medical, pharmacy and other relevant data using a plurality of machine learning structures by analyzing and extracting medical treatment pattern of a claimant and generate recommendations as to appropriate interventions.

### SUMMARY OF THE INVENTION

It is an object of the invention to provide a cloud-based, scalable, advanced analytics platform, and method for, for analyzing complex medical risk data and providing dedicated electronic trigger signals for triggering risk-related activities or providing expert-system based insights for medical risk-transfer. In addition, it is also an object to provide an automated claims risk score system and measuring system, inter alia, allowing to transform data into actionable insights and activating trigger signal triggering and/or signaling automated operation of connected devices and systems. It is a further object to develop an automated claims risk score measuring system able to address FWA issues, as discussed above.

According to the present invention, these objects are achieved particularly through the features of the independent claims. In addition, further advantageous embodiments follow from the dependent claims and the description.

According to the present invention, the abovementioned objects are particularly achieved by the scalable machine-learning-based medical system for processing and monitoring of complex, big medical data (BMG) and providing dedicated electronic detection signals triggered by measured and/or forecasted medical data pattern, wherein the system comprises data interfaces capturing complex, big medical data (BMG) as medical datasets associated with a plurality of individuals and wherein the medical datasets associated with an individual comprise structured and/or unstructured data, in that the machine-learning-based system comprises a core engine comprising a monitoring unit for real-time capturing and monitoring of the complex medical data sets, wherein first structured, digital tractable medical data are extracted by applying a predefined medical markup detection to the complex medical datasets, in that the machine-learning-based system comprises a machine-learning unit for an automated segmentation, clustering and classification of the complex medical datasets by generating second structured, digital tractable medical data, in that the machine-learning-based system comprises a claim risk modelling structure applying dynamically adapted, predictive claim risks modelling based on the structured, digital tractable medical data providing predictive claim risk measure values, and in that the core engine provides output signal indicating automated identification of emerging risks based on the dynamically adapted, predictive claim risks modelling, the emerging risks being associated with a portfolio of risk-transfers assigned to a plurality of medical datasets and individuals, respectively, and/or (ii) detected forecasted medical data pattern, and/or (iii) automated identification of inter-dependencies or links between individuals and different portfolios.

This dynamic and sophisticated claims risk predictive and/or measuring system utilizes Artificial Intelligence and Machine Learning capabilities to pull actionable insights out of the most complex sets of medical insurance data. It can be tailored to specific products, businesses and/or clinical rules and/or market needs. The system is able to analyze detailed medical claims data in real time, automatically detecting anomalies and outliers to mitigate FWA. Meanwhile, it automates and speeds up low risk claims, improving a possible customer experience and, most importantly, managing occurring claims development over time. The inventive claims risk score system allow to make the entire claims approval process transparent, more effective, and operationally efficient.

An advanced claims risk score system based on medical and clinical measuring data is implemented as a scalable machine learning device that, inter alia, uses normalized and clean medical claims data provided by an associated digital platform. The solution uses multiple structures and methods like trend, outlier, and network analysis on data from claims, healthcare providers, patients, and diagnostics. The inventive claims risk score system is able to draws upon carefully crafted Medical Key Performance Indicators (KPI) distilled from an inventive combination and technical selection of medical and/or clinical knowledge and advanced statistical treatment. These KPIs were developed to capture any abnormal trends and a technical network structure was put in place to capture subtle cases of abuse such as fraud rings. The KPI's are technical measures capturing complex processes and quantities, which are otherwise difficult to capture. The KPIs, inter alia, comprise
(i) KPI Diagnosis—Procedure: This KPI provides a measure for the average cost per day typically seen for different procedures across various diagnoses. Any claim seen with a daily cost abnormally higher than what is seen for the specific diagnosis-procedure combination would be owed to higher charges seen by medical service providers and doctors, and should be investigated further to see if the higher costs are warranted.
(ii) KPI Age: This KPI aggregates and provides a general claim cost measure seen across age bands for different diagnoses. If claims are incurred for an age band that doesn't generally pertain to a specific diagnosis, it needs to be checked further to identify any form of mistreatment or abnormal behavior.
(iii) KPI Diagnosis—This KPI flags specific diagnoses that are deemed to be risky by default. Claims with Diagnoses pertaining to critical illnesses are marked as claims to be flagged for further investigation. Customizability at each client level also allows this KPI to add a higher risk weightage to any diagnosis the client may want to always investigate further.

Finally, the impact of each claim, e.g. based on a financial impact measure, can be taken into account, allowing users to focus on the most important and relevant claims only. These indicators can technically be further enriched by clinical rule-based structures like procedure-diagnosis patterns. The claims risk score measuring device or system uses diagnosis and procedure coding to rank the claims based on clinical rule-based structures, which then identify the underlying abuse patterns and inconsistencies in the clinical process or cycle. This includes detected billing of unnecessary services, mismatch or unbundling of services, ordering excessive tests or supplies.

The combination of data analytics capabilities, integrated medical knowledge to define the appropriate clinical structures and an inputted broad risk expertise allows medical risk-transfer systems or insurance systems to get more value off of medical or clinical risk-related data, as e.g. also claim data, translating the insights into concrete actions that contribute to sustainable business growth. The inventive claims risk score measuring and/or predictive system is both a forward- and backward-looking device/tool as, by inter alia being triggered by and rely on historical data, the automated system/device provides the precise metrics to measure/assess new claims and generate an appropriate forward looking view. The inventive claims risk measuring structure is a dynamic and sophisticated risk measuring and assessment structure, inter alia using clinical, policy and business rule-based structures. It allows to be specifically tailored to specific requirements and allows to leverage and accumulate risk-related knowledge delivering multiple technical benefits, as e.g. being enabled to provide an appropriate expert system basis. It can help to automatically detect and/or reduce medical claims abuse and trigger optimization to a portfolio steering by enabling the identification of medical cost drivers and focus on high risk claims. Low risk claims go through an automated process supporting faster turnaround time and greater consistency in decisions and/or system signaling. Automated claims triaging help to improve machine-user interaction and thus user experience, to deliver efficiencies and technically reduce claims processing costs, which ultimately reduces operational costs of the automated system. In addition, the inventive claims risk measuring system has a modular design and can be easily extended to multiple claims dimensions. The inventive claims risk score measuring structure and system, is a technically scalable solution with different automation layers, which offers a holistic way to automatically detect claims abuse and to automatically optimize a portfolio by triggering and/or signaling an appropriate steering. Equipped with operation-driven KPIs measures, the used Machine Learning engine and an insightful front-end leverages the used broad risk knowledge and risk-transfer data, where the claims risk score measuring system allows to reduce analysis and operation time of the system, inter alia allowing real-time processing, and move from data analysis to direct, real-time signaling of actions or operations of connected automated system.

Further, the present inventive device and system is realized as a powerful new medical tool and device that puts medical data to be processed and analyzed automatically. The inventive cloud-based, scalable, advanced analytics platform analyses complex medical insurance data and delivers actionable insights for medical insurers. It leverages cutting-edge technology and a more than 150 years of risk knowledge to make sense of complex datasets and unveil essential information to better monitor and ultimately enhance the performance of your portfolio. The inventive system has the further advantage that it requires only minimum infrastructure investment, with short implementation time. Through the inventive system's technically reduced, simplified, and user-friendly interface, users can simply click to monitor portfolio level experience or to drill down into detailed medical claims analytics. The inventive system automatically, detects, recognizes, and senses trends, outliers, patterns, consumer behaviors and provides robust dynamic visualizations using Machine Learning techniques and built-in business rules as well as its predictive claims risk scoring model structure.

The inventive system comprises three main components: (A) the Experience Monitoring, (B) the Medical Claims Dashboard and (C) the system's Claims Risk Model data processing structure:

(A) Experience Monitoring

Making use of sophisticated algorithms, the inventive Experience Monitoring module identifies and detects emerging risks quickly, accurately, and reliably in real time. The digital platform has the capability to cross-reference complex customer-provider relationships across different claims datasets. It is equipped with a robust early warning mechanism that uses dynamic visualizations to highlight when future experiences differ from previously expected outcomes. Its interactive drill down functionality lets you immediately spot possible causes for deviations, so you can proactively apply corrective actions to mitigate losses or take steps to seize previously unforeseen latent opportunities.

(B) Medical Claims Dashboard

The system's medical claims dashboard with an automated intelligent assessment supports monitoring of medical claims by identifying medical cost drivers focusing on patterns, trends, and outliers, identifying, and flagging potential abusive cases and automatically detecting anomalies and outliers to reduce fraud, waste, or abuse. It also supports you in setting up a robust strategy for data processing of heterogenous hospital or medical data provider networks.

With the Experience Monitoring and the Medical Claims Dashboard, medical insurers can clearly identify emerging risks that need corrective actions or opportunities that can be exploited. The output enables medical insurance systems to accelerate and automated business decisions, reducing the arduous steps of manually pulling and interpreting data from multiple data sources. Technical key features are, inter alia:
Real-time portfolio monitoring: derives information from your medical portfolio in real-time, quickly identifying and spotting portfolio outliers, trends as well as faster claim cycles.
Advanced analytics structures: identifies potential high-risk claims and unveils cost of risk using simulation data, network visualizations, performance scorecards and predictive analytics.
Complex automated decisioning: ability to cross-reference multiple customer-provider relationships across claims in complex Big Data sets to optimize portfolio strategy decisions.
Machine-based intelligent assessment: advanced risk assessment such as profiling, pattern recognition to immediately spot anomalies.
Automated claims triage structures: timely and efficient claims automation, combining your existing infrastructure with advanced analytics, visualization, data security and a robust set of API's to simplify the integration.
Actionable expert-system insights: insights powered by machine learning and Swiss Re's more than 150 years of global insurance expertise and business knowledge help you build an insights-driven operating strategy.

(C) Claims Risk Score Model Structure

The implemented and used inventive Claims Risk Score Model structure is a dynamic and sophisticated claims risk model structure utilizing Artificial Intelligence and Machine Learning capabilities to pull actionable expert-system-based insights from the most complex and/or heterogenous sets of medical insurance data. It can be tailored to specific products, businesses and clinical rule structures and market needs. The inventive system's Claims Risk Score Model structure automatically analyses detailed medical claims data in real time, automatically detecting anomalies and outliers to mitigate Fraud, Waste or Abuse (FWA). Meanwhile, it automates and speeds up low risk claims, improving your customer experience and, most importantly, managing your claims development over time. The inventive system's Claims Risk Score Model structures makes an entire claims approval process technically transparent, more effective, and operationally efficient.

The inventive system's advantages, inter alia, comprise: (i) Augmented data insights capability: using sophisticated Machine Learning algorithms, Artificial Intelligence and scalable cutting-edge technology, the inventive system consolidates data from multiple sources and systems, and delivers invaluable actionable data insights and reports for medical insurers faster and with less effort; (ii) Optimized medical portfolio: the inventive system's integrated real-time analytics environment equipped with data visualization, data security and a robust set of APIs give medical insurers the possibility to identify and address threats or seize otherwise hidden opportunities, securing a more efficient claims workflow management and ultimately optimizing your medical portfolio; (iii) Better customer experience: by cross-referencing and making sense of all of their data stored enterprise wide, medical insurers can identify high-risk claims, speed up the processing of standard claims and free up time to create a more differentiated customer experience; and (iv) Competitive edge boosted by Swiss Re expertise: the inventive system offers medical insurers the possibility to tap into Swiss Re's over 150 years of risk knowledge, global exposure and industry expertise combined with the inventive system's superior monitoring and data analytics capabilities to optimize their portfolios and gain competitive edge.

### BRIEF DESCRIPTION OF THE DRAWINGS

The present invention will be explained in more detail below relying on examples and with reference to these drawings in which:

FIG. 1 shows a diagram, schematically illustrating a scalable machine-learning-based medical system 1 for processing and monitoring of complex, big medical data (BMG) and providing dedicated electronic detection signals triggered by measured and/or forecasted medical data pattern.

FIG. 2 shows a diagram, schematically illustrating a typical patient centric healthcare ecosystem from the perspective of big data with its significant stakeholders and their diversified data sources (structured/semi-structured/unstructured). The present invention also allows to capture the impact of big data in medicine and healthcare results by identifying new data sources such as social media platforms, telematics, wearable devices etc. in addition to the analysis of legacy sources that includes patient medical history, diagnostic and clinical trials data, drug effectiveness index etc. When the mixture of these data sources and analytics are coupled together, it provides an improved and extended source of information for health-care and medical data allowing to attain the inventive solution.

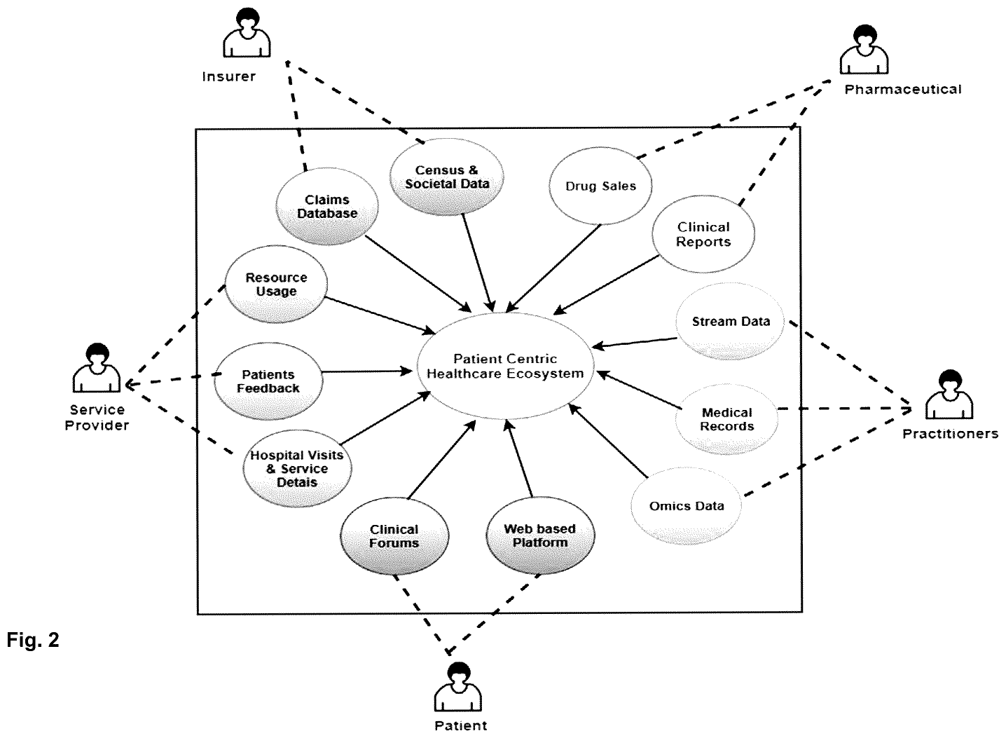

FIG. 3 shows a diagram, schematically illustrating some of the big medical data processing pipeline in a neuro-medical context, including data management, mapping, processing, interpretation, and inference detection. Large and complex clinical/medical datasets require data-specific analytic protocols for managing raw data, extracting valuable information, transforming the information to knowledge, and enabling decision-making and action that are evidence-based and/or risk-based. The present predictive data processing and analytics can be useful in all future state dependent inquiries or explorations. Anticipating future failures or systemic changes using multi-source data streams that generate hundreds or thousands of data points is critical in decision-making, whether when buying a stock, preparing for natural disasters, forecasting pandemics, projecting the course of normal or pathological aging or anticipating the behavior of social groups. The present inventive predictive data processing aims to uncover patterns and expose critical relations in phenomena using the associations between data elements detected in the observed process. Two types of predictive data processing techniques are proposed to realize the present invention: model-based or model-free. In addition to the inventive use of machine-learning structures, predictive time series processing can be use moving averages to build a model using historical or training data and extrapolate the trend predicted by the model into the future. Multivariate regression methods can be applied to represent variable interdependencies between predictors and responses in terms of some base functions (e.g. polynomials) whose coefficients capture the influence of all variables on the outcomes and facilitate forward predictions.

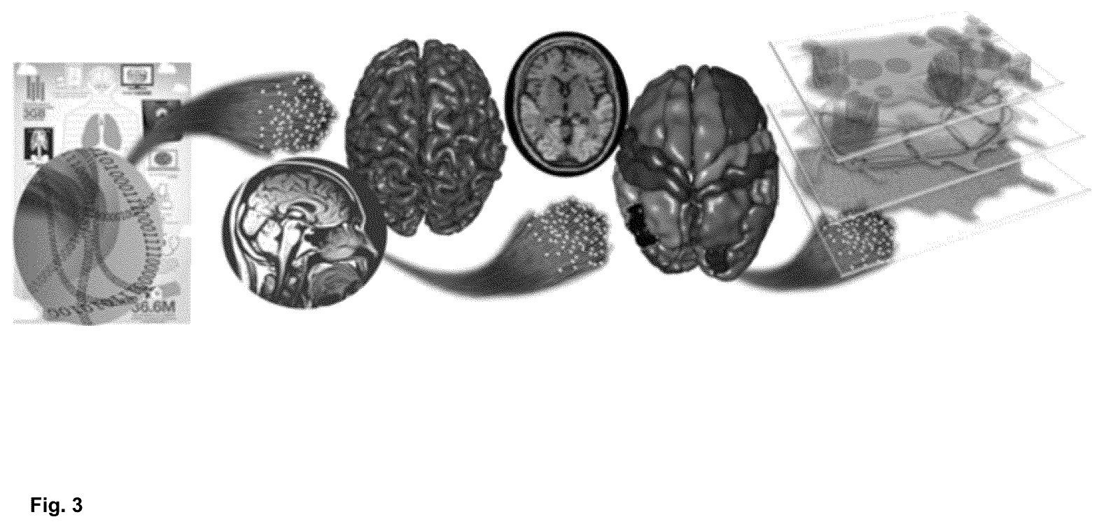

FIG. 4 shows a diagram, schematically illustrating an exemplary measured degree of anomaly across the KPI values. Due to the exponential design, the measured DA score is increasing very quickly for high values of the KPI score.

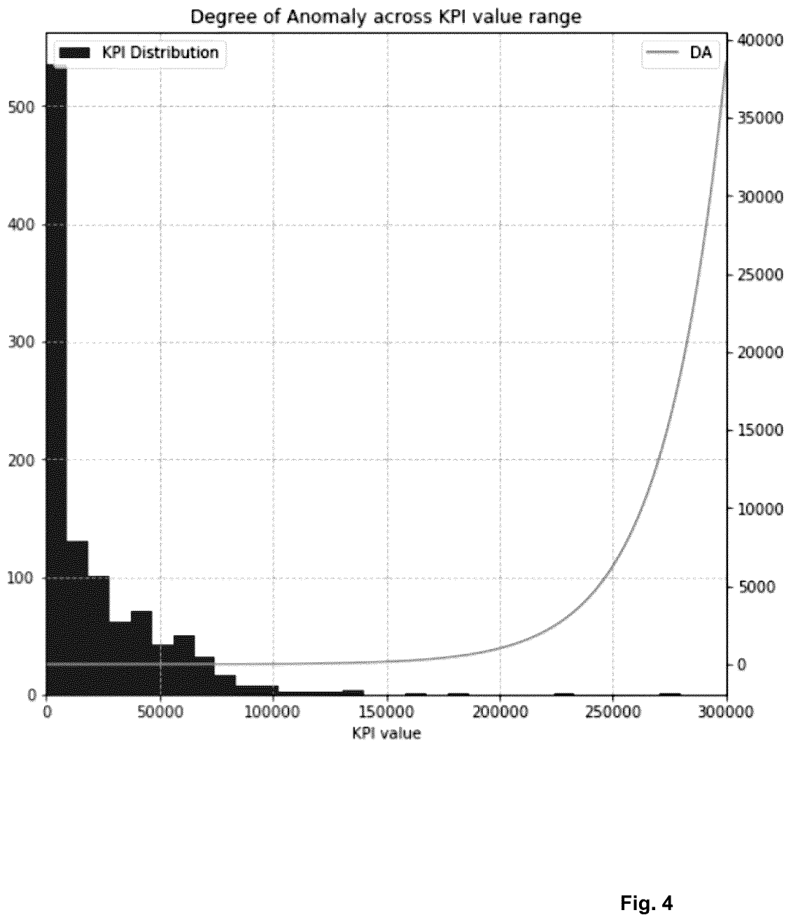

FIG. 5 shows a diagram, schematically illustrating an exemplary measured recency distribution for different diagnosis. FIG. 5 illustrates why a single value of threshold would not assess/measure the risk properly for all cases.

FIG. 6 shows a diagram, schematically illustrating an exemplary data processing pipeline of the claim risk score modelling. Initially, the basic preprocessing steps generate the basic scores and then the ranking and flagging structures provide the relevant results.

FIG. 7 shows a diagram, schematically illustrating an exemplary measured, i.e. incurred, cumulative sum value of claims costs across combined ranking based on a detected anomaly score measure.

### DETAILED DESCRIPTION OF THE PREFERRED EMBODIMENTS

The present invention is a cloud-based, digital platform 1 and/or anomaly detection system 1 for analyzing complex medical input data 101 and providing actionable insights for first medical insurance systems 2 and medical insurers as operators of first medical insurance systems 2. Through an interface 11, a user (insurer) is enabled to monitor medical portfolio data on portfolio level or by detailed medical claims analytics. The present invention is able to provide trends, outliers, patterns, consumer behaviors and provides robust dynamic visualizations using Machine Learning techniques and built-in business rules as well as an inventive predictive claims risk scoring model.

In particular, the present invention is an automated, scalable machine-learning-based medical system 1 for processing and monitoring of complex, big medical data (BMG) and providing dedicated electronic detection signals triggered by measured and/or forecasted medical data pattern. The measured and/or forecasted medical data pattern can at least comprise outliners 712 and/or anomalies 711 and/or significances 713 and/or variations detected by the system 1.

The system 1 comprises data interfaces 11 capturing complex, big medical data (BMG) 101 as medical datasets 811 associated with a plurality of individuals and wherein the medical datasets associated with an individual comprise structured and/or unstructured data. The structured and/or unstructured data can at least comprise image data and/or genetic data, and/or medical/healthcare data.

The machine-learning-based system 1 comprises a core engine 7 comprising a monitoring unit 81 for real-time capturing and monitoring of the complex medical data sets 811, wherein first structured, digital tractable medical data 8111, . . . , 8113 are extracted by applying a predefined medical markup detection to the complex medical datasets. As an embodiment variant, the predefined markup detection can be based on defined KPIs (Key Performance Indicator). One of the most important concepts to be understood is the technical problems associated with the application of the forward-backward looking structures. On the one hand there are the needs of the system 1 for the application of a forward looking structure. For instance, as soon as a claim is incurred and detected upon monitoring the measured medical data sets, the applied predictive modelling structure should be able to predict the associated risk, i.e. the probability to measure a frequency of occurrences of a predefined medical event providing a corresponding indication for a claim in a form of a score measure value or a trigger flag. On the other hand a single claim technically does not carry enough information for a reliable risk measure generation. In the inventive solution, it is the assessment based on historical data that will provide the correct metrics so that the system is able to evaluate a new claim against to. This fundamental component is, in the present solution, the applied backward-looking setup which can, for example, be realized by a statistical recognition engine. The statistical recognition engine of the system 1 can e.g. be dynamically operated, i.e. KPIs as measuring parameters can be dynamically adapted based on dynamically captured historical medical and/or claim data sets. Important information distilled from the historical medical and/or claim data sets together with proper risk KPI design technically permits for a proper automated data driven risk assessment. Technically, it is first the separation and then the combination of the forward and backward components in the present inventive solution that permits a predictive and functional claims risk score measure modelling and anomaly pattern recognition.

The machine-learning-based system 1 comprises a machine-learning unit 82 for an automated segmentation, clustering, and classification of the complex medical datasets 821 by generating second structured, digital tractable medical data 8211, . . . , 8213. The first structured, digital tractable medical data 8111, . . . , 8113 extracted by applying a predefined medical markup detection to the complex medical datasets, e.g. by applying the corresponding appropriately structured KPI measuring parameters build the specifically selected input parameters to the machine-learning unit 82 and/or claim risk modelling structure (83) of the system 1. The second structured, digital tractable medical data 8211, . . . , 8213 can e.g. comprise key drivers of the portfolio, the portfolio being the aggregated group of individuals and associated medical and/or claim data sets, wherein the key drivers can be automatically identified by the system 1 and the machine-learning unit 82, respectively. The key driver parameters can comprise e.g. key portfolio drivers and/or key medical cost drivers automatically detected by the system 1. The second structured, digital tractable medical data 8211, . . . , 8213 can also comprise KPIs or at least parts of the defined KPIs. The machine-learning-based system 1 comprises a claim risk modelling structure 83 applying dynamically adapted, predictive claim risks modelling 832 based on the structured, digital tractable medical data providing predictive claim risk measure values 8311, . . . , 8313.

The dynamically adapted, predictive risks modelling structure 832 comprises e.g. an unsupervised machine learning algorithm. The modelling structure 832 creates distributions for the defined Performance Indicators or KPIs (Key Performance Indicator). It is to be noted, that performance indicators, as defined in this application, are used and defined as a technical measures, which are contributed by the technical skilled man put an appropriate real world measuring link to the system 1, thus in order to rely the realization of the automation on measurable and technically reproducible measuring quantities. The KPIs' definitions have nothing to do with a probably underlying business method and does not contribute or optimize such a probably underlying purely administrative business method but is needed for the technical realization of the automation. Thus, a performance indicator or key performance indicator (KPI), as used herein, is a type of physical performance measurement. The KPIs' measuring values provide a measure for the achieved performance level of a complex physical system, which can also comprise organizational units, or of a particular activity (such as processes, projects, programs, or products) in which its operation is involved. Key performance indicators are technically defining a set of values against which to measure. These raw sets of values, which can be measured or otherwise captured to the system 1, are aggregated by the system 1 and are called indicators or indicator measures. There are two categories of measurements for KPIs. Quantitative measures can be measured with a specific objective numeric value measured against a standard. Usually quantitative measures are not subject to distortion, personal feelings, prejudices, or interpretations. Qualitative measures are intended to measure non-numeric conformance to a standard, which can even represent not “per se” technical measures as levels of personal feelings, tastes, opinions, or experiences. Such qualitative measures must be interpreted or projected by the system 1 against a standard scale or index measure. Thus, the technical requirements to measure such qualitative measures are scalability and projectability/mappability. Such machine-based interpretation can e.g. be realized by a further ML- or AI-based unit. It is to be noted, that such an “indicator” can only measure what has happened, in the past tense, so the only type of measurement is descriptive or lagging. Any KPI that attempts to measure something in a future state as predictive, diagnostic, or prescriptive is not defined herein as an “indicator”, but as a “prognosticator” measure, which can be measured using simulation or predictive modelling structures, e.g. based on the measured KPIs.

Again, the herein used KPI measures are technically fundamental components to the problem of automation of risk-measure triggered electronic signaling, in particular to automated claim risk handling. The technical concepts developed here, although related to medical risks and medial claim risks, i.e. the measurable probability of occurring aggregated claim in a future time window, are fundamental enough so to be applied in many other fields of automation. In the context of the present invention, it is fundamental to understand the forward-backward looking structures and the KPIs as extracted measuring metrics. As mentioned, there are the needs in the technical field of automation for appropriate forward looking solutions. For instance as soon as a claim is detected by the system 1, the modelling structure should be able to predict and/or forecast and/or generate an associated risk measure providing a corresponding indication for a claim in a form of a score or a flag. On the other hand a single claim does not carry enough information for a risk measure generation. It is only the measurements based on historical data that will provide the correct metrics so that a new claim can be measured against to. This fundamental, but not visible component is the backward-looking setup which can e.g. be realized within a statistical engine. Important information distilled from the historical claims data together with proper risk KPI design permits for a proper data driven risk assessment. It is first the separation and then the combination of the forward and backward components that permits a functional claims risk score measurement.

Regarding the KPIs and their tree structure, as soon as the forward-backward claims handling structure and setup is established, the construction of KPIs that assess the risk can technically take place. The first step is the KPI construction itself. There are two main construction classes for the present application: (i) Statistical measures driven KPIs. In some cases a statistical measuring score can be used. E.g. how many standard deviations away from its measured historical mean value, a treatment cost of a single claim lays; (ii) Risk-transfer driven KPIs. In this case KPIs are directly derived from the risk-transfer structure. For example the KPI of readmission. Readmissions are the cases where for one risk-transfer for the same diagnosis a user claims multiple times. Although the structure may accept this in some cases, in many others can be an indication if an abuse.

The next step is the construction of these KPIs within the different dimensions of a claims dataset. At a first look, it could be assumable that there is no specific hierarchical structure but soon connections will emerge. For instance, the readmission KPI measures can be used both for a doctor or insured member. Moreover the readmissions can be related to diagnosis or to other procedures/surgeries. The present solution proposed to technically capture the underlying hierarchical structure by a tree structure. The branches of the tree are the claim risk measuring dimensions (e.g. doctors, medicines, diagnosis) while the leaves are the KPIs themselves (e.g. readmission, claim recency). Starting by this technical approach, this allows to develop and extend the different KPI measures across the different claim dimensions in a way that also instructs an easy engineering development. Despite the fact that the individual KPIs score measures hold very interesting information, they cannot directly provide risk measures. It is a statistical mapping linked to those KPIs which allows to measure the risk, i.e. provide a risk measure. The computation of a deviation in respect to an expected, i.e. forecasted, value can be done in many ways. For instance a nonparametric quantile scoring can be applied as a starting point. Nevertheless, for the needs of the present invention, a more vigorous treatment is introduced.

To provide an automation structure capable to come from measured KPIs to measured risk values, the data processing structure and data processing steps are now explained that take place from the construction of the claims KPIs until their expression as risk.

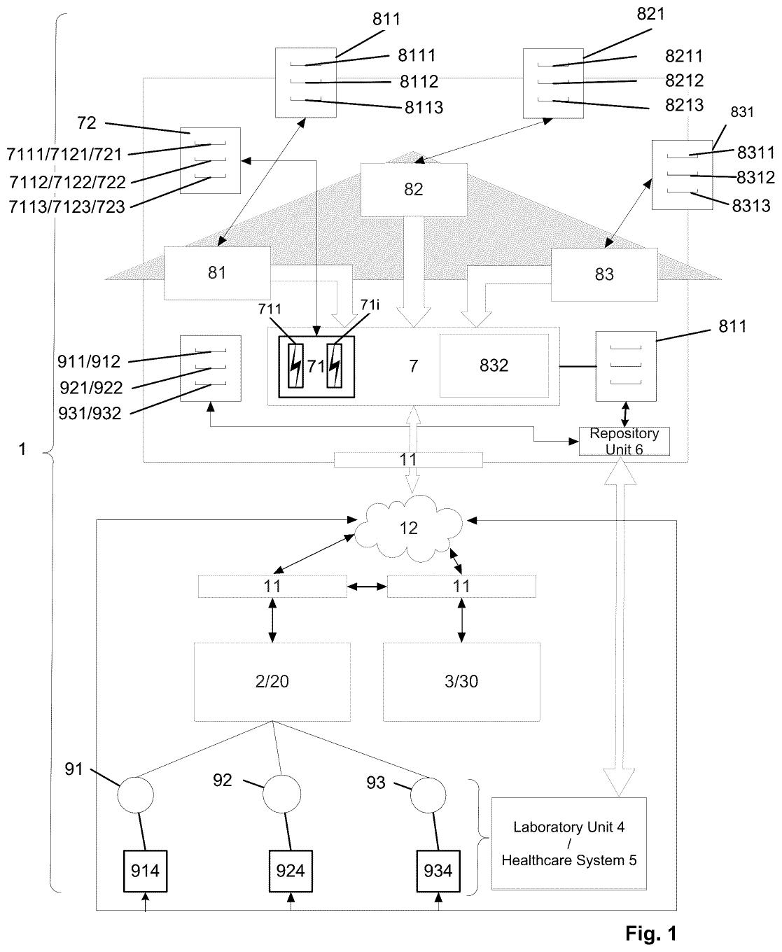

To measure a degree of anomaly, the deviation is measured as a degree of anomaly (DA) score measure. As shown by relation 1, the DA score is constructed so that a given value of the iₜₕ KPI xᵢ is compared against a measured mean value i of the historical KPI distribution normalized by the standard deviation i of this distribution. Moreover, as illustrated in FIG. 1, this computation appears in an exponential factor and the measured score increases very rapidly according to the underlying deviation so that any interesting cases will surface quickly high up.

$$D A ( {x}_{i} ) = {e}^{\frac{{x}_{i} - {μ}_{i}}{{σ}_{i}}} \qquad ( relation 1 )$$

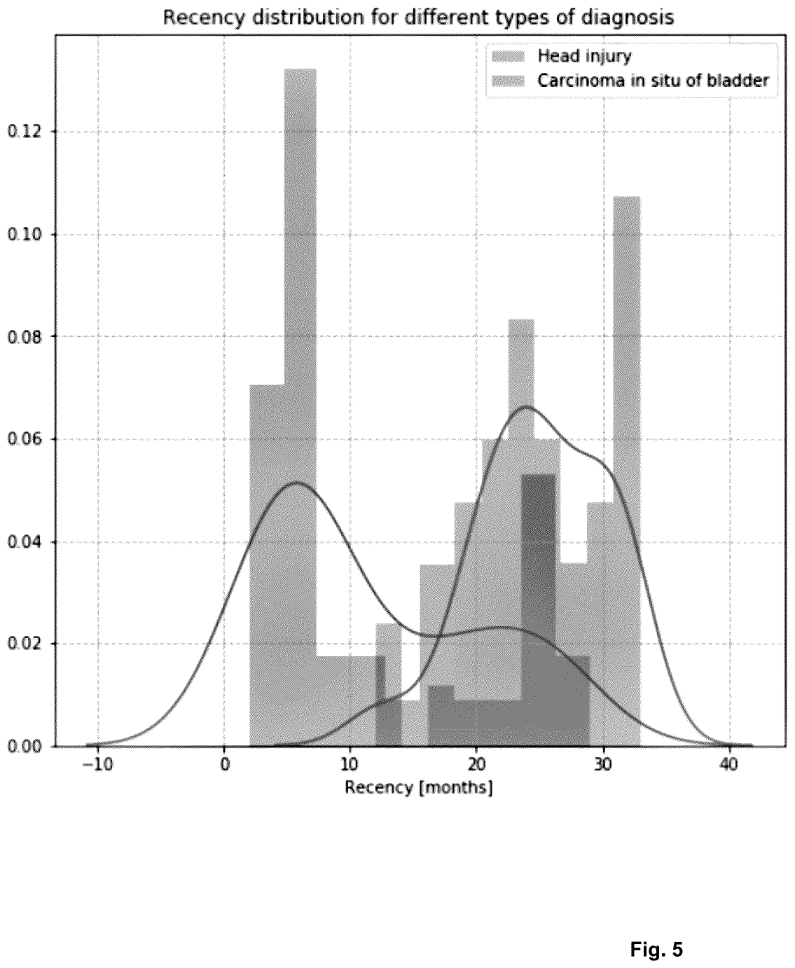

In order to construct the DA, each KPI should be accompanied with the statistical measures of mean and standard deviation. This information is returned from the historical claims data in the backward looking setup structure. Moreover in case a KPI being dependent in some claim dimension (e.g. diagnosis) the final rule is not a single threshold but an adjusted threshold to each case. Like this any final rules remain as robust as possible. FIG. 5 illustrates this dynamic threshold mechanism.

Apart of the DA measure for the individual KPIs, the system 1 is capable of measuring and providing a total score measure considering all the individual KPIs of a claim together. For this purpose, a composite degree of anomaly (CDA) is constructed as shown by the relation 2, where i refers to the iₜₕ KPI and n is the total number of KPIs. Due to the fact that the DA of the individual KPIs is following an exponential form, the composite degree of anomaly (CDA) can be measured by just summing up the measured individual components. Like this it is ensured that all DA contribute equally in the CDA and at the same time any extreme outlier will drive the CDA score even if it comes from only one KPI.

$$C D A ( x ) = \frac{1}{n} {\sum }_{i = 1}^{n} DA ( {x}_{i} ) \qquad ( relation 2 )$$

Having computed the DA and CDA scores one can easily derive the relative ranking and flag outputs. These outputs are directly related with insurance risk and are the ones typically support claims inspections.

TABLE 1
From KPI score measure to the degree of Anomaly

| KPI_LOS | LOS_MEAN | LIS_STD | LOS_DA |
|---|---|---|---|
| 8 | 5 | 8.513121 | 1.422473 |
| 4 | 3 | 30.16221 | 1.03371 |
| 11 | 7 | 3.210808 | 3.475687 |

Although many times forgotten in automatic claims assessment and measuring/forecasting, monetary impact measures may be a desired component which, when properly integrated in the output, can provide a powerful measure within the resulting signal. At first place, a financial impact KPI is defined or constructed. The relation follows the same logic of DA adapted for the financial score measure. Like this all low value claims have the same minimum score while the expensive ones pick up their value rapidly in an exponential fashion. After that, a financial ranking score R_{f} can be generated across the claims based on this score.

In a second step when the financial ranking is accomplished, it can be used to be integrated with other claims rankings R_{C} (e.g. composite ranking based on CDA). This can be achieved using the geometric mean of the two rankings of relation 3 where Rᵢ=1=R_{f} and Rᵢ=2=R_{C}. As a result a final combined ranking is in place to properly order the claims and further simplify the analysis efforts of the user.

R_{Cf}=(πᵢ₌₁ⁿRᵢ)^{1/n} (relation 3)

The degree of combination of the different KPIs and DA described above, can offer multiple levels of automation and final uses of the output signaling of the system 1. Although the number of automation levels is difficult to be explicitly defined, the awareness of their existence is important for a final data processing structure of the system 1. In the following, three main automation levels are identified and discussed:
1. Individual KPIs: The output of the individual KPI measures can be very important. It is often a technical prerequisite for claim assessment and predictive processing and many times the result that any client/user is interested to. As an example, an abnormally high pricing of a concrete surgery in a claim can itself trigger, e.g. by thresholding, a red flag signalizing further inspection.
2. Combined KPIs: The assessment of multiple KPIs in once in many cases can be even more required than the measurement of the individual ones. In such cases the CDA score is generated (relation 2) or a global outlier detection algorithm is employed. Such generation allow to detect interesting marginal cases that might not have been spotted when assessing the individual scores measures. In addition it has the extra advantage that it provides a higher level of automation especially for cases when the number of KPIs is big.
3. Outlier Monetary Impact: There is a possibility for a monetary or financial-based impact measure incorporation. For instance, there can be a claim with a very high DA measure but on the same time of a very small, claimed value. In order to take both into account, the DA score and financial score measure are convoluted using a geometric mean method (see above).

TABLE 2
Rankings and Flags. Example of single KPI outputs together with
the global flags and the composite rankings and combined rankings

| KCY KPI | Comp. R | RCY Flag | Global Flag | Comp. Rf |
|---|---|---|---|---|
| 142884 | 1 | FALSE | True | 1 |
| 142885 | 1910 | FALSE | True | 2 |
| 219133 | 228626 | FALSE | FALSE | 3 |
| 120712 | 122021 | FALSE | FALSE | 4 |

Apart of the assessment and measuring of the KPIs and the relevant DA that support ranking and flagging, other metrics can be required, too. For instance metrics that measures trends or network characteristics of the dataset can contribute highly to the assessment. Despite the fact that such metrics will not take part in the decision layer of a concrete claim assessment, they can perfectly provide insights that deal with other claim entities. For instance they might provide insights to support blacklisting of a specific doctor or an inspection of a clinic. Specific details in medical data are further discussed below.

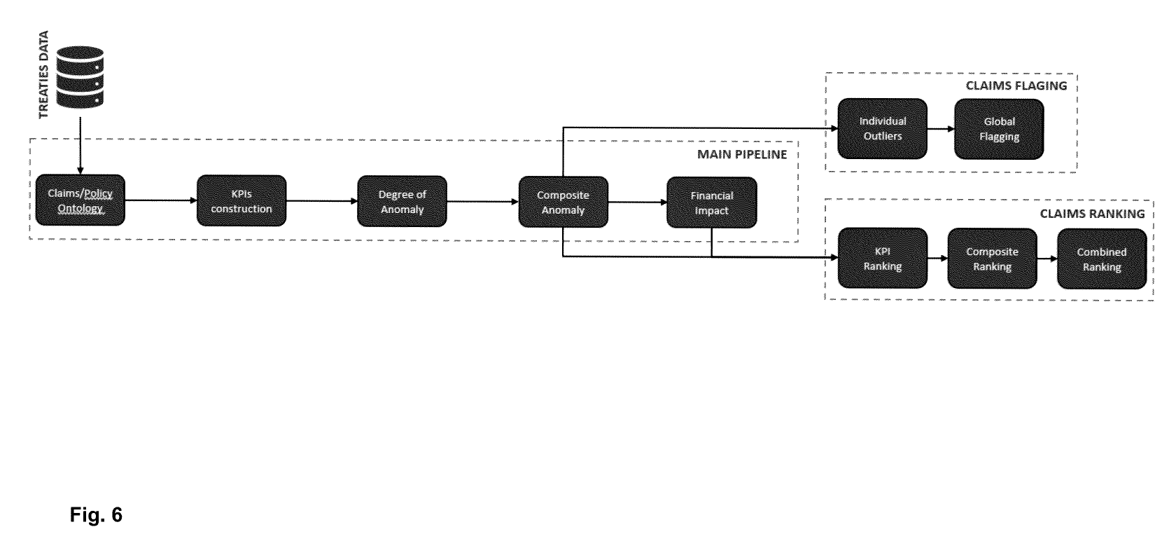

The fundamental technical concepts, developed above, are now used to illustrate an embodiment variant of a claim risk scoring model structure for the concrete case of a medical claims dataset. The various modeling steps are illustrated in FIG. 6 and the details are explained below.

The principles of KPI construction were show above. Here a detailed list of the KPIs is illustrated:
Recency: Recency is simply defined by how many days after the inception date a claim has occurred. Claims with recency of a small number, especially if they are coupled with a severe diagnosis are to be further checked.
Length of Stay: Length of stay (LOS) is the number of days a person spends in a hospital. Claims with a high number of LOS are typically double checked.
Readmission: Readmission counts the number of times a claim with the same diagnosis is sent by the same insured person. Going here a step further, the readmissions are divided with the median of their time intervals so that the same number of readmissions that happen too often have higher score (equal to higher risk) comparing to readmission that happen spread across time.
Complexity: It assigns a medical complexity score to the insured person that claim. The score is constructed by multiplying the number of distinct diagnosis and the number of distinct diagnosis groups.
Price Anomaly Measure: It assigns a score that reflects the anomaly in terms of price of the specific diagnosis with respect to an average value of this diagnosis. Following the medical KPI construction step, the DA and the CDA can be measured/assessed.

One of the most important outputs for a claims assessment can be a final ranking of the anomalies measured and/or observed in a claims dataset. There are three layers of ranking outputs:
1. KPI Measure Ranking: For each of the medical KPIs developed above, there is a relevant ranking in the claims dataset. The ranking is an index following the KPI DA score.
2. Composite Measure Ranking: Developed in a similar way like the KPI ranking above, it takes in to account multiple KPIs in once using the CDA.
3. Composite Financial or Monetary Measure Ranking: It considers together the composite ranking and the financial ranking.

These three-level output signaling aim to satisfy all assessment requirements. In its basic use, one can trust the fully automated structure and solution, while at the same time if needs appear, one can navigate through composite to individual KPI rankings and get the most detailed insight measures.

Although the inventive claim measures ranking is very precise, it is to be noted that there is no such thing like a limit of inspections. And this is done by design. A ranking structure will only generate a ranking for all the input claims. If a historical data assessment structure is used where thousands of claims have to be assessed, the output will contain all of them in a decreasing in terms of interest order. The question that follows is where the inspection limit is.

The answer cannot be a simple number because the inspection limit is defined by the resources and risk capacities of the risk-transfer system or the user. For instance, if a risk-transfer user, as e.g. an insurance system client, is able to use a lot of manpower to review and inspect the claims, a big number of top ranked cases will be considered. In the opposite case of an insurance client with few resources may only the top 20 or top 20 claims will be considered.

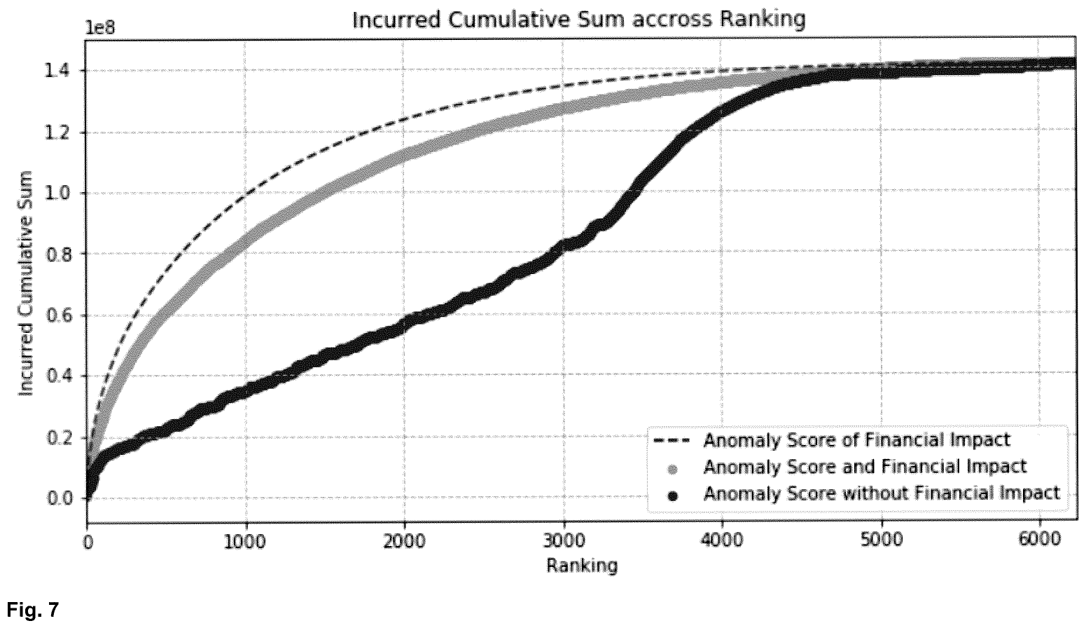

In order to support the risk-transfer system or the insurer as operator of the risk-transfer system with this operation, a rank versus the total loss output can be provided. In this output the rank list is accompanied with the relevant cumulative or aggregated sum of the financial losses, see FIG. 7. The trend is as expected picking up quickly in the beginning (due to the financial impact component in the rank) and reaches soon a plateau. Like this a powerful tool is provided to drive the investigation limits.

Apart of the ranking, another highly desirable output signaling of claims assessment is an appropriate and automated claims flagging. Often a risk-transfer system requires a list of claims applied to a modelling structure to get back the relevant flags for each claim. Typically in such output signaling, a new information of claim flag is in place where, for example, green means “nothing to be checked” while red means “the claim should be further investigated”. Having precomputed the DA scores the outlier detection task can now be performed by the inventive system 1. As in the case of ranking, the system 1 can provide multiple flagging outputs:
1. KPI Outliers: The outliers are assessed for each individual KPI. Since it is a straightforward task for satisfying explainability criteria, a simple nonparametric statistical driven method can e.g. be employed. The Tuckey interquartile method can e.g. be used based on the eq. T=Qᵤ+k(Qᵤ−Qₗ) with k=1.5.
2. Global Outliers: The outliers are found considering all the KPIs DA together. The inputs are the DA scores and the method used is the isolation forest. It is a novel method based on binary decision trees and eventually ensembles which are made by averaging all the trees in the forest.

One of the most important assessment capabilities on top of historical claims data relates the trend detection and/or recognition. The breakdown of costs is the first result to look at, but many times even more important is how these costs evolve in time. In order to detect important trends, five trend KPIs are constructed (see results in table 5):
Total Claims Increase: The percent increase per year in terms of number of claims.
Total Cost Increase: The percent increase per year in terms of total costs.
Total Insured Members Increase: The percent increase per year in terms of insured members.
Cost per Claim Increase: The percent increase per year in terms of cost per claim.
Claims per Insured Increase: The percent increase per year in terms of number of claims per insured member.

For the above KPI measures, proper data cohorts take place. For instance the claim percentage cost increase is a KPI generated for a specific year and a specific diagnosis. Moreover the structure is realized in a way that different claims dimension combinations can be taken into account. Currently the output of the trends insights includes results of year-diagnosis and year-diagnosis doctor. If needed, result outputs can be expanded and other combinations like year-diagnosis-hospital can take place.

Due to the nature of the highly interconnected claims data, there is a broad and unexplored search space for detecting and automated recognizing interesting patterns. This opportunity arises not by assessing at the individual data points, but rather by exploring the rich directional and transitive connections that link them. The dataset is represented as a large graph where nodes represent doctors, hospitals, insured members etc. and edges represent claimed services. On top of this dataset network analysis techniques can be applied. Generally speaking there are two main categories of graph analysis techniques:
Network structure techniques: In this category the global structure of the network is considered, and one can search for communities that share abnormal behavior or tight-knit communities that show anomalous aggregated statistics. Unfortunately these methods require a proper graph DB in place supported with novel network libraries. Due to the lack of such technical environment, this approach is not considered here.
Ego-net approaches: This second category is simpler in construction and deals with statistics based on individual nodes and node's local neighborhood. In this analysis three ego-net KPIs are constructed. Given a node n and its local neighborhood N, the system 1 can derive the
   degree: the number of nodes |N| in the neighborhood
   weight: The total number of claims that a node is associated with
   entropy ratio: How evenly the node associates with entities in its neighborhood, in terms of number of claims (or claim amount). For the calculation the relation 4 is used where p k is the percentage of the n t h node interaction with the neighbor k out of its total operation. With the help of the summation term one derives the entropy which is finally normalized with the first term so to belong in the [0; 1] range.

$$E {R}_{n} = \frac{1}{log \vert N \vert} {\sum }_{k ∈ N {p}_{k}} log ( \frac{1}{{p}_{k}} ) ( relation 4 )$$

TABLE 3
Example output of ego-network KPI calculations

| doctor | PR. diag. | degree | weight | Eclaims | Eamount |
|---|---|---|---|---|---|
| 0 | J124 | 4 | 6 | 0.89 | 0.950754 |
| 0 | J820 | 2 | 2 | 1 | 0.871483 |
| 4 | H102 | 2 | 4 | 1 | 0.999957 |
| 10 | C018 | 5 | 5 | 1 | 0.968104 |
| 13 | D043 | 2 | 2 | 1 | 0.999268 |

TABLE 4
Example output of generated rankings and flags for captured claims

| Composite_Rf | KPI_PRICE_R | KPI_LOS_R | Composite_R | KPI_PRICE_FLAG | KPI_LOS_FLAG | Global_Flag |
|---|---|---|---|---|---|---|
| 1 | 1 | 146 | 1 | TRUE | FALSE | TRUE |
| 2 | 60 | 636 | 80 | TRUE | FALSE | TRUE |
| 3 | 5 | 598 | 55 | TRUE | FALSE | TRUE |
| 4 | 6 | 599 | 57 | TRUE | FALSE | TRUE |
| 5 | 9 | 113 | 63 | TRUE | FALSE | TRUE |
| 6 | 2 | 597 | 5 | TRUE | FALSE | TRUE |
| 7 | 278 | 98 | 81 | TRUE | FALSE | TRUE |
| 8 | 306 | 115 | 69 | FALSE | FALSE | TRUE |

TABLE 5
Example output of detected trend KPI measures

| Pr. | | | | | | |
|---|---|---|---|---|---|---|
| diagnosis | year | N_units_incr. | Members_incr. | Amount_incr. | CostsPerUnit_incr. | UnitsPerMember_incr. |
| A081 | 2016 | 35.41666667 | 37.58865248 | 43.30749361 | 5.827072207 | −1.578608247 |
| A081 | 2017 | 11.79487179 | 2.577319588 | −26.30021621 | −34.07588147 | 8.985955418 |
| A081 | 2018 | 100 | 92.46231156 | 40.36097163 | −29.81951418 | 3.916449086 |
| A081 | 2019 | −26.14678899 | −27.154047 | −27.69356931 | −2.094398192 | 1.382723357 |

As more data is added to the modelling structure 832, the modelling structure 832 recreates distributions for each of defined KPIs (Key Performance Indicator) to take into account newer claim behavior. Based on this, the model learns from the new claims to generate new thresholds based on which claims can then be scored in real-time.

The core engine 7 provides output signals 72 indicating automated identification of emerging risks 7111, . . . , 711i based on the dynamically adapted, predictive claim risks modelling 832, the emerging risks 7111, . . . , 711i being associated with a portfolio 911, . . . , 931 of risk-transfers 912, 922, 932 assigned to a plurality of medical datasets (811) and individuals, respectively, and/or (ii) detected forecasted medical data pattern, and/or (iii) automated identification of inter-dependencies or links between individuals and different portfolios.

Thus, the present invention consists of three main components: (i) the Experience Monitoring module, (ii) the Medical Claims Dashboard and (iii) the system's Claims Risk Model data processing structure:
(i) The Experience Monitoring module, or monitoring unit 81, identifies emerging risks in real time. The platform has the capability to cross-reference complex customer-provider relationships across different claims datasets. It is equipped with an early warning mechanism that uses dynamic visualizations to highlight when predicted experiences differ from previously expected or historic outcomes. The inventive system allows to monitor possible causes for deviations, so that a user can proactively apply corrective actions to mitigate losses or take steps to seize previously unforeseen latent opportunities.
(ii) The Medical Claims Dashboard, or claim risk modelling structure 83, allows to monitor medical claims by identifying medical cost drivers focusing on patterns, trends, and outliers, identifying, and flagging potential abusive cases and automatically detecting anomalies and outliers to reduce fraud, waste, or abuse. It also allows to support user to set up a robust strategy for the insurers hospital or medical provider network.
(iii) The Claims Risk Score Model data processing structure, or machine-learning unit 82, comprises a dynamic claims risk modelling based on artificial intelligence (AI) and machine learning (ML) capabilities to pull actionable insights from complex sets of medical insurance data. It can be tailored to insurers products, business and clinical rules and market needs. The inventive system's Claims Risk Score Model analyses detailed medical claims data in real time, automatically detecting anomalies and outliers to mitigate Fraud, Waste or Abuse (FWA). The inventive system further allows to automate and speed up low risk claims, improving the customer (insurer) experience and, most importantly, managing the claims development over time. The inventive system's Claims Risk Score Model data processing structure allows to make the entire claims approval process transparent, more effective, and operationally efficient.

The scalable machine-learning-based system 1 can e.g. realized as a cloud-based, digital platform providing via a graphical user interface (GUI) automated actionable expert-system insights into portfolio trends by detecting occurring anomalies and/or optimizing areas swiftly for timely corrective actions. The system 1 can e.g. provide a dynamic portfolio optimizer via a cloud-based, digital platform. The system 1 can dynamically provide indications for optimized claim triage to a user. The system 1 can e.g. provide automated identification of multiple possible relationships of individuals across different claims. Further, the system 1 can comprise a medical claims dashboard providing navigation and monitoring of claims of a specific portfolio by a user.

For processing and monitoring of the complex, big medical data (BMG), the system 1 can at least comprise additionally to the machine learning structures or artificial intelligences structures, built-in business rules and/or predictive claim risk scoring modelling.

In particular, for the medical data processing of the system 1, a medical data processing pipeline can be applied at least comprising (i) an extraction unit extracting first structured, digital tractable medical data from the captured medical datasets by raw observations extraction and/or data aggregation and/or data scrubbing and/or semantic mapping, and/or (ii) an information generation unit generating second structured, digital tractable medical data by data fusion and/or statistical sum up and/or data fusion and/or second stage data processing, and/or (iii) knowledge generation unit generating maps and modelling structures and/or causal interference and/or network analytics and/or linkages and relations, and/or (iv) action generation unit generating digital indications for actionable decisions and/or treatments and/or forecasted or predicted cause of a disease and/or predicted healthcare outcome and/or predicted claim occurrences associated with an individual.

The medical data processing pipeline comprises machine-learning structures and/or classification structures and/or and network analytics providing unsupervised data mining, hierarchical clustering, pattern recognition, fuzzy clustering and/or trend identification for the captured and/or measured medical datasets. A predictive analytics structure can e.g. be applied to uncover patterns and expose critical relations in phenomena using the associations between data elements of an observed process detected in the captured and/or measured medical datasets. As an embodiment variant, a Generalized Logistic (GL) structure can e.g. be applied that scales data uniformly to an appropriate interval by learning a generalized logistic function to fit the empirical cumulative distribution function of the data. The inventive application of the GL-structure can be proved to be very effective; it is intrinsically robust to outliers, so it is particularly suitable for diagnostic/classification modeling in the present medical application where the number of samples can also be small; it scales the data in a nonlinear fashion, which leads to potential improvement in accuracy.

It is to be noted that machine-based predictive modeling has huge technical potential because of its ability to generalize from data. Even though predictive modeling, proposed herein, lack the skills of a human expert, it can handle much larger amounts of data and can potentially find subtle patterns in the data that a human cannot. However, machine-based predictive modeling relies heavily on training data and are dependent on data quality. Ideally, a model should extract the existing signal from the data and disregard any spurious patterns (noise). Unfortunately, this is not an easy task for medical data, since medical data are often far from perfect; some of the imperfections include irrelevant variables, small numbers of samples, missing values, and outliers. Therefore, data preprocessing is useful to be applied in the present invention in order to increase the ability of the machine-based predictive modeling to extract useful information. There are various approaches targeting different aspects of data imperfection; such as imputations for missing values, smoothing for removing the superimposed noise, or excluding the outlier examples. Then, there are various transformations of variables from common scaling and centering of the data values to more advanced feature engineering techniques, which can be applied. Each of those techniques can make a significant improvement in predictive modeling performance when learned on the transformed data.

In the proposed, inventive machine learning and data mining process, data scaling and data normalization refer to the same data preprocessing procedure, and these two terminologies are used interchangeably herein; their aim is to consolidate or transfer the data into ranges and forms that are appropriate for the applied modeling and mining. It is to be noted, that modeling structures applied herein and trained on scaled data usually have significantly higher performance compared to the models trained on unscaled data, so data scaling should be regarded as an important step in the used data preprocessing. Data scaling is particularly important for the present inventive system and method since it uses distance measures for some of the KPI measures, such as nearest neighbor classification and clustering. In addition, artificial Neural Network modeling requires the input data to be normalized, so that the learning process can be more stable and faster.

The embodiment variant using a GL-structure for data scaling is adapted from the histogram equalization technique, and it can map both the original and future data into a desired interval. The algorithm has no assumption on the sample distribution and utilizes generalized logistic functions to approximate cumulative density functions. Since it maps data into a uniformly distributed range of values, the points that were previously densely concentrated on some interval become more discernible, which allows more room for representation of the subtle differences between them. In addition, applying a GL-structure reduces the distance of outliers from other samples, which makes the modelling robust to the outliers. This technical advantage is particularly significant in present diagnostic/classification modeling based on medical datasets, where the number of samples can be small, and outliers have a huge impact on the model training, leading to poor accuracy.

In the present solution, the values of a variable in the samples are modeled as a random variable (r.v.) X. In the applied GL-structure, the scaled value v′ of a value v, is obtained by v=P_{X}(v), with P_{X}(⋅) is the applied cumulative density function (CDF) of the r.v. X. Using a CDF as a mapping can also be applied for the Histogram Equalization technique. The difference of the applied GL-structure to the Histogram Equalization technique is that not only the CDF is used in the present embodiment variant to scale the data, but also learn/approximate the functional expression of the CDF, so that it can be used to scale unseen values.

From the medical data, the exact functional form of the needed cumulative density function (CDF) of a variable whose value is represented by the r.v. X is typically not known; therefore, as an embodiment variant the CDF can be approximated. An empirical cumulative density function (ECDF) can be found by using the relation

$${\overline{P}}_{X} ( v ) = \frac{1}{n} {\sum }_{i = 1}^{n} {1}_{{x}_{i} \leq v} \qquad ( relation 4 )$$

with P̅_{X}(v) is the ECDF at a value v, n is the number of medical data samples, and xᵢ is the value of the variable in the i^{th} medical data sample or dataset.

However, in many cases, the ECDF has no functional form expression for the medical datasets. Moreover, the original data tend to be noisy, so the ECDF is usually difficult to be applied. Therefore, the inventive solution proposes to apply a generalized logistic (GL) structure to approximate the ECDF. It can be shown that a logistic function can be used to accurately approximate the CDF of a normal distribution. For the proposed application of the GL-structure, there is no need to make any assumption on the distribution of the data; therefore, a more general form of the logistic function, called the generalized logistic (GL) structure is presently applied

$$L ( x ) = \frac{1}{{( 1 + {Qe}^{- B ( x - M )} )}^{1 / v}} ( relation 5 )$$

Compared to the logistic function structures, sometimes used in prior art systems, the proposed application of the above discussed GL-structure provides the flexibility to approximate a larger variety of distributions. One of the notable properties of the structure based on the relation 5 is that it maps the values in the interval (∞,−∞) to the interval (0,1). This property makes the proposed GL-structure technically robust to outliers and guarantees that the scaled data will be in the measuring interval of (0,1).

It is to be noted, that during the medical data collection period, the data might be corrupted for various reasons; e.g., system error, human error, sample contamination, etc. Therefore, a data de-noising or outlier detection procedure can be necessary in the data preprocessing step. The herein proposed GL-structure is intrinsically capable of handling situations where there are noisy samples and outliers in the samples. For the inventive system 1, it can be shown for situations if there are no outliers in samples, all applied medical data scaling structures perform similarly. However, when an outlier exists in the data, the original values in the normal range are squeezed after the scaling. So it is a technical advantage that the outlier's impact to the applied GL structure is typically neglectable, as can be shown. Outliers are samples deviate strongly from the measured majority of (normal) samples, so the number of outliers will be always much smaller than the number of normal samples, and therefore, the contribution of outliers to the CDF of the samples is neglectable. However, outliers do not necessarily need to be the result of measurement errors, but may also occur due to variability, and represent completely valid instances. There are situations that can be particularly concerned with such anomalies in the captured medical datasets as they may carry valuable information about some rare modality of the processes responsible for its generation. For such applications, as a further embodiment variant, algorithms for outlier detection can be applied to interrogate the data and bring the focus to the rare signal in the data, and the applied medical data preprocessing structure can be less appropriate to use for such purposes. Nevertheless, regardless of the outliers' origin (error or variability), for the automated supervised task of classification, outliers are typically detrimental for classification accuracy, and their removal/correction can be recommendable.

To handle missing data within the captured and/or measured medical datasets a two-step process can be applied comprising a first step of deleting a medical dataset for the data processing by the system 1 if data are detected to be missing completely at random indicating the probability of an observation being missing is the same for all individuals, and a second step of modelling, imputing and/or correcting for the missing data to obtain unbiased inference if the pattern of data missingness is detected to be not completely at random, comprising when non-response rates are different in different subpopulations resulting in a variable probability of observing such an individual. For modelling the data missingness a logistic regression structure can e.g. be applied, in which the outcome variable equals 1 for observed cases or 0 for unobserved entities. When an outcome variable is missing at random the system 1 can e.g. exclude the missing data as unobserved, wherein all data affecting the probability of missingness comprising characteristics of an individual and/or subject demographics are controlled by the applied regression modelling structure.

### LIST OF REFERENCE SIGNS

1 Scalable machine-learning-based medical system
101 Complex medical input data (BMG)
2 First medical insurance system (primary insurance system)
20 First electronically automated resource-pooling system
21 Payment transfer modules
211, 212, 213 First risk transfer parameters
221, 222, 223 First payment parameters
22 Secured data store
3 Second insurance system (reinsurance system)
30 Second electronically automated resource-pooling system
31 Payment transfer modules
311, 312, 313 Second risk transfer parameters
321, 322, 323 Second payment parameters
32 Secured data store
4 Laboratory Unit
5 Healthcare System
6 Repository unit
7 Core engine
71 Trigger module
711 Trigger for anomalies
7111, 7112, 7113 First trigger parameters
712 Trigger for outliners
7121, 7122, 7123 Second trigger parameters
713 Trigger for significances
7121, 7122, 7123 Third trigger parameters
714 Trigger for variations
7121, 7122, 7123 Forth trigger parameters
72 Data store
721, 722, 723 Defined risk events
8 Core engine
81 Monitoring Unit
811 Medical datasets each associated with an individual
8111, . . . , 8113 First structured, digital tractable medical data
82 Machine-learning unit
821 Medical datasets each associated with an individual
8211, . . . , 8213 Second structured, digital tractable medical data
83 Claim risk modelling structure
831 Datasets with claim risk measure values
8311, 8312, 8313 Claim risk measure values
832 Dynamically adapted, predictive claim risks modelling
9 Total risk exposure
91, 92, 93 Risk exposed individuals
911, 921, 931 Portfolio of risk-transfers
912, 922, 932 Risk-transfers of a portfolio
913, 923, 933 Individual risk of a risk exposed individual
914, 924, 934 Capturing or measuring devices
915, 925, 935 Laboratory individual-specific parameters
916, 926, 936 Self-declaration parameter
917, 927, 937 Occurred losses at a risk exposed individual
918, 928, 938 Relative risk factor for a risk exposed individual
10 Table with retrievable stored risk classes with assigned risk class criteria
101, 102, 103 Risk classes
110, 111, 112 Risk class criteria assigned to risk classes
121, 122, 123 Class category parameters
131, 132, 133 Class category criteria
11 Data interface
12 Data transmission network

1. A scalable machine-learning-based medical system and/or anomaly detection system for processing and monitoring complex, big medical data and providing dedicated electronic detection signals triggered by a measured and/or pattern-recognized medical data pattern, the system comprising:
data interfaces configured to capture the complex, big medical data as medical datasets associated with a plurality of individuals, the medical datasets including structured and/or unstructured data;
a machine-learning unit configured to process the complex, big medical data; and
a core engine including a monitoring unit for real-time capturing and monitoring of the medical datasets, wherein
first structured, digital tractable medical data is extracted by applying a predefined medical markup detection to the medical datasets, the predefined medical markup detection extracting the first structured, digital tractable medical data by applying key performance measuring parameters as extracted measuring metrics from historically captured medical datasets to the medical datasets providing a forward-backward looking structure,
the machine-learning unit is configured to perform an automated segmentation, clustering, and classification of the medical datasets by generating second structured, digital tractable medical data taking the first structured, digital tractable medical data as input parameters,
the system further comprises a claim risk modelling structure configured to apply dynamically adapted, predictive claim risks modelling based on the second structured, digital tractable medical data to provide predictive claim risk measure values,
the core engine is configured to provide output signals indicating automated identification of emerging risks based on the dynamically adapted, predictive claim risks modelling, the emerging risks being measurable probability values of occurring aggregated claims in a future time window, being at least associated with the medical data pattern, and being associated with a portfolio of risk-transfers assigned to a plurality of the medical datasets and the individuals, and
a degree of anomaly is measured by a deviation measured by comparing a given value of the ith key performance measuring parameter xi against a measured mean value i of a distribution of historically measured key performance measuring parameters normalized by a standard deviation i of the distribution.

2. The system according to claim 1, wherein the predefined medical markup detection at least partially includes structured measuring parameters which are extracted and built depending on a forward-backward structure realized by the machine-learning unit and/or the claim risk modelling structure.

3. The system according to claim 2, wherein the structured measuring parameters are extracted from the medical datasets by a statistical recognition engine and/or pattern detection engine capturing historical medical datasets for the recognition.

4. The system according to claim 3, wherein
the statistical recognition engine and/or pattern detection engine dynamically extracts the structured measuring parameters as applied measuring metrics, and
the historical medical datasets are dynamically updated.

5. The system according to claim 2, wherein the structured measuring parameters at least comprise a recency and/or a length of stay and/or a readmission and/or a complexity and/or price anomaly measuring parameter.

6. The system according to claim 2, wherein the system generates and signals an electronic flagging dependent of key performance measuring parameter outliner and/or global outliner thresholding.

7. The system according to claim 1, wherein the machine-learning unit and/or the claim risk modelling structure includes a Generalized Logistic structure scaling data uniformly to an appropriate interval by learning a generalized logistic function to fit an empirical cumulative distribution function of the medical datasets.

8. The system according to claim 1, wherein the system is realized as a cloud-based, digital platform providing, via a graphical user interface, automated actionable expert-system insights into portfolio trends by detecting occurring anomalies and/or optimizing areas swiftly for timely corrective actions.

9. The system according to claim 1, wherein the medical data pattern at least includes outliners and/or anomalies and/or significances and/or variations detected by the system.

10. The system according to claim 1, wherein the structured and/or unstructured data at least includes image data and/or genetic data and/or medical/healthcare data.

11. The system according claim 1, wherein, for the processing and the monitoring of the complex, big medical data, the system at least includes machine learning structures or artificial intelligence structures and built-in business rules and/or predictive claim risk scoring modelling.

12. The system according to claim 1, wherein the system provides a dynamic portfolio optimizer via a cloud-based, digital platform.

13. The system according to claim 1, wherein the system dynamically provides indications for optimized claim triage.

14. The system according to claim 1, wherein the system provides automated identification of multiple possible relationships of the individuals across different claims.

15. The system according to claim 1, further comprising a medical claims dashboard providing navigation and monitoring of claims of a specific portfolio by a user.

16. The system according to claim 1, further comprising a medical data processing pipeline,
wherein the medical data processing pipeline at least includes:
(i) an extraction unit extracting the first structured, digital tractable medical data from the medical datasets by raw observations extraction and/or data aggregation and/or data scrubbing and/or semantic mapping, and/or
(ii) an information generation unit generating the second structured, digital tractable medical data by data fusion and/or statistical sum up and/or data fusion and/or second stage data processing, and/or
(iii) a knowledge generation unit generating maps and modelling structures and/or causal interference and/or network analytics and/or linkages and relations, and/or
(iv) an action generation unit generating digital indications for actionable decisions and/or treatments and/or forecasted or predicted cause of a disease and/or predicted healthcare outcome and/or predicted claim occurrences associated with an individual.

17. The system according to claim 16, wherein the medical data processing pipeline comprises machine-learning structures and/or classification structures and/or and network analytics providing unsupervised data mining, hierarchical clustering, pattern recognition, fuzzy clustering and/or trend identification for the medical datasets.

18. The system according to claim 1, wherein a predictive analytics structure is applied to uncover patterns and expose critical relations in phenomena using associations between data elements of an observed process detected in the medical datasets.

19. The system according to claim 1, wherein, to handle missing data within the medical datasets, a two-step process is applied, the two-step process including:
a first step of deleting a medical dataset for the processing if data are detected to be missing completely at random indicating a probability of an observation being missing is the same for all individuals, and
a second step of modelling, imputing, and/or correcting for the missing data to obtain unbiased inference if a pattern of data missingness is detected to be not completely at random, including when non-response rates are different in different subpopulations resulting in a variable probability of observing such an individual.

20. The system according to claim 19, wherein for modelling data missingness a logistic regression structure is applied, in which an outcome variable equals 1 for observed cases or 0 for unobserved entities.

21. The system according to claim 19, wherein
when the outcome variable is missing at random, the system excludes the missing data as unobserved, and
all data affecting a probability of missingness including characteristics of an individual and/or subject demographics are controlled by an applied regression modelling structure.
